(19) United States
(12) Patent Application Publication
(10) Pub. No.: US 2003/0129584 A1
(43) Pub. Date: Jul. 10, 2003

(54) EVALUATION OF BIOLOGICAL AGENTS IN LIVING TARGET CELLS

(76) Inventor: Manuel Vega, Vigneux-Sur-Seine (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/168,075

(22) PCT Filed: Dec. 13, 2000
(86) PCT No.: PCT/FR00/03503

(30) Foreign Application Priority Data
Dec. 16, 1999 (FR) 99/15884

(51) Int. Cl. C12Q 1/70; C12Q 1/68; G01N 33/53; G06F 19/00; G01N 33/48; G01N 33/50

(52) U.S. Cl. 435/5; 435/6; 435/7.1; 702/20

(57) ABSTRACT

The invention concerns a method for screening and/or evaluating the performance of a set of biological agents, such as a library of recombinant viral or non-viral vectors, of recombinant proteins or antibodies in living target cells (complex biological system).

The invention relates to methods for screening and/or assessing the performance of a collection of biological agents, such as a library of recombinant viral or nonviral vectors (vectors for transferring genes), of vaccines, of recombinant proteins or of antibodies, in living target cells (complex biological system).

Progress relating to the transfer of genes in gene therapy depends on the ability to develop vectors which enable a substance to be expressed at the level of the target cell, with said substance having a positive therapeutic effect at the level of said cell. It is therefore important to have available vectors which are of clinical quality and which can be used in phase I trials.

The assessment parameters which are proposed in the literature for obtaining quantitative information with regard to the potential performance of a gene transfer vector are:

the titer of physical particles (pp) (Mittereder et al., J. Virol., 1996, 70, 11, 7498-7509; Atkinson et al., NAR, 1998, 26, 11, 2821-2823; Kechli et al., Hum. Gene Ther., 1998, 9, 4, 587-590; Nelson et al., Hum. Gene Ther., 1998, 9, 16, 2401-2405), which represents the total content of vector particles; as a rule, this titer is assessed either on the basis of the nucleic acid content of the vectors (hybridization of the nucleic acids or OD₂₆₀ in the case of AAV and AdV, respectively), or on the basis of the content of viral proteins (RT activity and content of p24, for example, in the case of MLV and HIV, respectively); physical measurement of the viral particles or of the genomes suffers from the drawback of being able to be confused with the presence of defective particles (defective-interfering particles or DI), and

the titer of infectious particles (ip: infectious units, transduction units) (Mittereder et al., loc. cit.; Weitzman et al., J. Virol., 1996, 70, 3, 1845-1854; Salvetti et al., Hum. Gene Ther., 1998, 9, 5, 695-706) is assessed by studying the changes which are observed in the infected cells (viral replication, integration of the provirus, cell lysis, expression of the transgene), using methods which are essentially based on serial dilutions followed either by a linear extrapolation or by an asymptotic approximation; thus, ip measures the number of particles which are active in the method whose effect is measured: ip does not correspond, therefore, to all the particles which are potentially active; ip constitutes a part of the physical particles (pp), with the other part of said physical particles consisting of inactive particles (nip or non-infectious particles),

detecting plaques in the case of lytic viruses; while this method is quantitative, it is difficult to implement and cannot be applied to noncytopathic viruses, and

varying the particles/infectivity ratios.

For example, in order to solve the problem of determining the titer and comparing different recombinant viruses used in gene therapy, the article by E. M. Atkinson et al. (NAR, 1998, 26, 11, 2821-2823) and international application PCT WO 99/11764 propose a new method which is regarded as being more reliable than those previously used and which essentially employs a step of amplifying the viral genetic material in a host cell line, preparations of standard vector of known titer, which preparations are obtained by serial dilutions, and an internal control of known titer. More specifically, the method comprises using a viral preparation to infect cells in different wells in a microtitration plate, replicating the viral genome in said host cell, chemically lysing said cell, hybridizing the nucleic acid and then determining the relative quantity of viral nucleic acid which has been replicated in each well.

In the article by A. R. Davis et al. (Gene Therapy, 1998, 5, 1148-1152), it is considered that optimum use of recombinant adenoviruses in gene therapy involves developing a technique which is suitable for rapidly separating new recombinants which are not contaminated by the wild-type virus. In order to do this, the authors of this article propose creating recombinant adenoviruses by cotransfecting 293 cells with the viral DNA derived from the 3′ region of the genome of a recombinant which does not comprise the E1 region but which expresses the fluorescent protein (GFP or green fluorescent protein) and a plasmid carrying the 5′ region of the genome; in this way, the cotransfection can be visualized by fluorescence microscopy.

The methods of the prior art resort exclusively to measuring the titer of physical particles (pp) and/or to measuring the titer of infectious particles (ip) for the purpose of assessing a gene transfer vector. The vector preparations having a high titer of infectious particles and a low physical particles/infectious particles ratio are regarded as being of high quality, with these two parameters being considered to supply quantitative information with regard to the performance of a gene transfer vector.

However, the procedures which are currently employed for assessing pp and ip vary depending on the type of vector, are not particularly reproducible and are imprecise; furthermore, these parameters are not sufficiently informative to make it possible to precisely define the characteristics of a vector and therefore to assess its performance.

Consequently, the object given to the present invention is that of providing a standardized method which is capable of assessing the interaction between a gene therapy vector, and, more generally, any biological agent whatsoever, and a complex biological system (living target cells).

Another object of the invention is to provide a method for screening a collection of complex biological agents with a view to selecting the biological agent which is most suitable for the sought-after application.

The present invention relates to a method for assessing the performance of a collection of complex biological agents in living target cells with which said biological agents interact, which method is characterized in that it comprises at least the following steps:

(a) preparing, for each biological agent in said collection, a range of samples, which range is obtained by making serial dilutions of said biological agent at a concentration R1,

(b) incubating each sample of said range of dilutions obtained in (a). with said target cells at a constant concentration R2,

(c) determining the product P of the reaction R1+R2, at a time t, in each of said samples,

(d) constructing a theoretical curve H from said experimental points R1 and P for each biological agent by iterative approximation of parameters which reflect the reaction R1+R2→P, at said time t, in accordance with the following equation:

P=Pₘₐₓ(πR1)^{r}/(κ+(πR1)^{r}) r=1, . . . , n (2).

in which:

R1 represents the concentration of biological agent in a sample of the range,

P represents the product of the reaction R1+R2 at a time t,

Pₘₐₓ represents the maximum capacity of the reaction,

κ represents the resistance of the biological system, at a constant concentration R2, to responding to said biological agent (constancy of resistance of R2),

r represents a coefficient which depends on R1 and which corresponds to the Hill coefficient, and

π represents the intrinsic power of the biological agent R1 to induce a response in the biological system (production of P at time t), and

(e) sorting the values of κ and π obtained in (d) for each biological agent and classifying the biological agents in accordance with said values.

The present invention also relates to a method for screening a collection of modified complex biological agents (library of mutants) in living target cells with which said biological agents interact, which method is characterized in that it comprises at least the steps (a) to (e) as defined above and a step (f) of selecting the biological agent which is most suited for the sought-after application.

According to an advantageous embodiment of said method, the collection of modified biological agents comprises a library of mutants which is obtained by naturally or artificially introducing one or more mutations into the nucleotide and/or peptide sequence of said biological agents.

Within the meaning of the present invention, a mutation is understood as being an insertion, a deletion or a substitution of at least one nucleotide or of at least one amino acid.

The methods according to the invention consist in analyzing the response of the biological system (production of a product P at a time t), for each range of biological agent to be tested, on the basis of the Hill equation.

The Hill equation is a general formalization which describes the interaction between different molecules. It expresses the quantity of product formed as a function of the concentration of reagents and of the affinity constant of the system.

Originally developed for studying dissociation between hemoglobin and oxygen, the Hill equation encompasses analysis of enzyme kinetics by the Michaelis-Menten equation, analysis of ligand-receptor binding and the analysis of allosteric systems.

Thus, in accordance with Hill, in the case of a simple reaction:

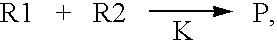

in which the affinity K between R1 and R2 changes in dependence on their concentrations; the Hill equation describes the accumulation of the product P as a function of the concentration of one of the reagents (R1 ) and of the intrinsic properties (K) of the system.

$$P = {\sum }_{r = 1}^{r = n} {P}_{max} \cdot {R1}^{r} / {( K + R1 )}^{r} ( R2 constant ) \qquad ( 1 )$$

in which R1, P, Pₘₐₓ and K respectively represent the concentration of the reagent R1, the concentration of the product P, the maximum capacity of the reaction and the affinity constant between R1 and R2.

The Hill coefficient r is a function of R1. r is equal to 1 when independent interactive binding sites are involved between R1 and R2, as in the case described by Michaelis-Menten. r varies from 1 to n for systems in which the sites involved in the interaction between R1 and R2 are not independent of each other. The affinity for R1 at the level of any R2 binding site varies as a function either of the degree of occupation of the other R2 sites or of the concentration of R1 itself or of the concentration of other regulators (positive or negative).

These analyses based on the Hill equation are still limited to individual proteins or to simple systems implemented under defined experimental conditions in a very precise manner.

Up to the present time, it was not possible to conceive that it would be possible for the Hill equation to be applied to the interaction between complex systems such as living cells and complex biological agents such as viruses, recombinant viral and nonviral vectors, such as gene transfer vectors, vaccines, recombinant proteins or antibodies, for example.

Surprisingly, the inventor has now found that it is possible to analyze complex systems, such as the infection of a cell with a virus or a gene therapy vector, by selecting parameters which are directly derived from the Hill equation.

Initial Definitions

R1 corresponds to the concentration of biological agent; in the present invention, it can signify, depending on the context, all the concentrations which are obtained by diluting the preparation of biological agent and which are used for determining product P or the biological agent itself;

biological agent is understood as meaning, by way of example but not in a limiting manner, a recombinant viral or nonviral vector containing a nucleic acid molecule of interest (gene, cassette for expressing a protein, antisense RNA molecule, ribozyme, recombinant viral genome or fragment of this genome) such as a gene transfer vector or an expression vector, a virus, an antibody, a vaccine or a recombinant protein;

R2 corresponds to the concentration of the living target cells; in the present invention it can signify, depending on the context, the concentration of the living target cells (constant) which is used for determining the product P or the living target cells themselves;

living target cells are understood as meaning target cells in vivo, in vitro or ex vivo, before they are modified by a biological agent;

P (output) represents the response of the target cells R2 to each dilution of biological agent (input) at a concentration R1; the product P can be determined either directly or indirectly by measuring biological parameters which reflect the response of the biological system (target cells) to said biological agent (or R1+R2 reaction or biological method); it is a matter, in particular, of measuring an enzymic activity, the expression of a transgene, the productivity of a virus, cytotoxicity, tumorigenesis, immunogenicity, etc. In this case, the biological test which is used for determining P is either an in vitro test or an in vivo test; it makes it possible to determine the biological parameters which are representative of the response of the biological system to the biological agent being studied;

the techniques used for determining, assessing, analyzing or calculating values of P at a time t are, in a non-limiting manner, measurements of radio-activity, of fluorescence, of luminescence, of absorbance or of cell counting;

the parameter π: π measures the intrinsic capacity of the biological agent for producing P in the living target cells under consideration; π is opposed to κ (resistance constant), which constitutes the factor of opposition of said cells to the production of P; for example, in the case in which the biological agent is represented by infectious viral particles (R1), it is possible to consider that, for each infectious viral particle added, the activity of the virus is given by the equation πR1; in order to obtain a response by the living target cells (production of P), the intrinsic capacity π should be greater than κ in the cell. π constitutes a specific characteristic of the biological agent being studied; in this context, variants of a biological agent being studied will not exhibit the same value of π in a given reaction method. It may be considered that π constitutes a parameter which reflects chemical activity as opposed to concentration in the case of simple chemical compounds. π constitutes a correction factor which affects the concentration R1 of the biological agent in order to indicate its power or true activity in a given reaction method; variations in π affect the equation (2) by displacing the curve toward the right or toward the left depending on whether the value of π decreases or increases; the slope of the curve obtained at step (d) increases when π increases; the curves which are obtained in step (d) which only differ from each other as far as the parameter π is concerned are not parallel to each other; π is a key parameter for characterizing the biological agent and determining its performance in accomplishing the reaction (biological method) being studied: π can be applied directly and practically in optimizing and developing the biological agent employed, to the extent that this parameter makes it possible to compare the relative power of variants of said agent; however, this parameter does not make it possible, on its own, to assess all the system;

the parameter κ (resistance constant): κ measures the internal resistance of the living target cells to the biological process induced by the biological agent for obtaining P; κ is a specific characteristic of the particular biological process (reaction between R1 and R2) and of the living target cells (cell type) being tested: the same biological process will lead, in the case of different cell lines or cell types, to different κ parameters being obtained; furthermore, the factors which have an influence on the performance of a cell in carrying out said biological process, such as contaminating agents or toxic agents, modify the value of κ for said cell; it is possible to consider that κ is analogous to the dissociation constants or affinity constants for simple biological reactions or chemical compounds; the variations in κ affect the equation (2) by displacing the curve from the right to the left depending on whether κ increases or decreases; the curves obtained in step (d) which only differ from each other as far as the parameter κ is concerned are parallel to each other; κ is a key parameter for appraising the performance of the biological test which is selected for assessing the overall reaction (reaction R1+R2); κ can be applied directly and practically in developing and validating the test which is selected for assessing the reaction in which the biological agent is involved and in assessing the susceptibility or the sensitivity of different cell types for participating in said reaction.

Surprisingly, in accordance with the method according to the invention, different complex biological agents and/or different living target cells can be compared and classified on the basis of their performance as assessed by at least the two abovementioned parameters, which are designated by the expression “Hill parameters”.

In this way, it actually becomes possible to precisely analyze and compare the biological response of the living target cells, both in vitro and in vivo, to complex biological agents.

Also in accordance with the invention, a certain number of parameters, which are derived from the Hill equation, can be recorded and used for quantifying the relevant characteristics of a complex system: biological agent, target cells and process or reaction resulting from their interaction.

For example, in the case in which the complex system is represented by the infection of a cell with a virus, it is possible to observe a large number of interactions (protein/protein, protein/nucleic acid, protein/small molecule) which, according to the invention, are capable of being described by the method which makes use of the Hill equation. The overall reaction, formalized by the reagents which enter in (input), namely the viruses and the cells, and the products of the reaction (output) [cellular response to infection] can be analyzed by using the Hill equation, with this being possible whatever the number of intermediate steps.

According to an advantageous embodiment of said methods, the biological agent is selected from the group comprising viruses, viral and nonviral vectors, vaccines, antibodies and recombinant proteins.

Complex reactions, such as those involving the inter-action between recombinant viruses (R1) and living cells (R2), and which induce a biological response, can be analyzed, and the reagents (R1 and R2) characterized, by the method according to the invention making use of the Hill equation (parameters π and κ) and, where appropriate, at least one of the derived parameters, as defined below.

According to another advantageous embodiment of said methods, P is determined in step (c) either directly, for example assaying P, or indirectly, for example using a biological test which is appropriately selected for measuring at least one parameter or one variable which reflects the response of the living target cells to said biological agent, as specified above.

According to another advantageous embodiment of said methods, they additionally comprise measuring at least one of the following derived parameters:

the overall efficiency ε of the reaction induced by the biological agent on said system,

the apparent titer τ of the biological agent,

the absolute titer θ of said biological agent, and

the heterogeneity index η of said biological reaction.

Definitions of These Derived Parameters

overall efficiency ε: ε measures the maximum overall efficiency of the reaction of the biological agent (R1), which is characterized by a given parameter π, with the living target cells (R2), which are characterized by a given parameter κ: ε is therefore specific for the biological agent (π)/biological system (κ) pair as far as the reaction being studied is concerned; changes in the parameters π and/or κ lead to changes in the parameter ε. ε is a key parameter for characterizing the efficiency of the overall reaction employing R1 and R2; it is particularly important and useful for optimizing the test when π and κ have been selected and for separately studying the changes of either π or κ;

the apparent titer τ: when R1 increases in the Hill equation (2), r increases from 1 to 2, 3, 4, etc. and P approaches its maximum value Pₘₐₓ. In the other direction, R1 is only able to decrease to a minimum point (R1ₘᵢₙ) to which the minimum values of r and P correspond. The sigmoidal Hill curve is not symmetrical: only its right arm is asymptotic (up to Pₘₐₓ); on its left arm, the curve has an origin at R1ₘᵢₙ. From a biological point of view, the fact that no P exists for R1<R1ₘᵢₙ means that there is no reaction product when the concentration of biological agent is <R1ₘᵢₙ: the system does not respond to values of R1<R1ₘᵢₙ. R1ₘᵢₙ therefore represents the minimum quantity of R1 which induces a response in the living target cells in question and is represented by τ; the titer, defined in this way, does not correspond either to an asymptotic value or to a value approached by extrapolation but, instead, to a precise parameter of the Hill equation, and corresponds to the mathematical origin of the theoretical curve obtained in (d) . It is therefore possible to consider that τ measures the limiting dilution or the apparent titer of the biological agent being studied; the value of τ is determined by the sensitivity limit of the system and by the method used for measuring the product P; it is for this reason that it is designated apparent titer; τ is specific for the quantity of biological agent tested and represents the apparent concentration of the biological agent; it is expressed in units per volume (maximum dilution of biological agent which induces production of P). In other words, τ is represented by the maximum R1 for which the Hill coefficient r reaches its minimum value, with said Hill coefficient becoming constant for a value equal to or close to 1. τ according to the present invention corresponds to the titer which is generally used for viruses, antibodies and vectors; however, contrary to what is described in the prior art, this parameter on its own does not make it possible to assess a complex biological system. The variations in τ affect the equation (2) by displacing the curve toward the right or to the left depending on whether the value of τ decreases or increases, respectively. τ constitutes a key parameter which measures the apparent concentration of the initial supply of biological agent which is required for the use which it is desired to make of it;

absolute titer θ: θ measures the absolute titer of biological agent; the value of θ is neither determined by, nor dependent on the sensitivity limit of the target cells tested or the method used for measuring P; this is why θ is designated absolute titer; θ is specific for the initial quantity of biological agent tested; it represents the true physical concentration of biological agent and is expressed in units per volume (i.e. the maximum dilution of biological agent which induces the production of P); θ is obtained in accordance with the following equation (3):

θπ=τ/s (3)

in which s represents the sensitivity of the detection method.

Consequently, in the case of biological agents which are assessed using the same method for detecting P, the following expression, which is represented in the following equation (4), is valid:

θ1π1/ι1=θ2π2/ι2=θnπn/ιn=constant (4).

It is possible to use equation (4) to obtain the ratio between two absolute titers, corresponding to two different biological agent preparations, from the values of π and τ for these two biological agent preparations. The variations of θ affect the equation (2) by displacing the curve toward the right or toward the left and/or by changing the slope of the curve. θ is the parameter which measures the initial absolute concentration of biological agent.

The heterogeneity index η: η measures the internal heterogeneity of the reaction, which can be due either to the cells (discontinuity in the resistance constant κ) or to the biological agent (discontinuity in the intrinsic power π). The presence of an internal heterogeneity in the reaction R1+R2 can be detected by the appearance of plateaus in the development of the Hill coefficient, corresponding to the Hill curve which fits the experimental data. η is therefore defined as the heterogeneity index and its value corresponds to the number of plateaus observed in the development of the Hill coefficient: one plateau: η=1; two plateaus: η=2; three plateaus: η=3; n plateau: η=n. η is a key parameter for analyzing the reaction in detail. It is useful for studying each plateau which is identified. The presence of plateaus is expressed by an abrupt discontinuity in κ or π. Complex processes, such as those assessed in the present invention, consist of a succession of events in a multidimensional network of biological reactions which are interlinked and interregulated. Thus, the resistance constant κ for a particular reaction is a macroscopic indicator of the overall resistance of the biological reaction (κ=κ₁×κ₂×κᵢ . . . κₙ). If the contribution of the microscopic resistance constants (κ₁, κ₂, . . . κᵢ, . . . κₙ) for the individual plateaus involved in the reaction were homogeneous, and if no threshold existed for passing from one plateau to another, no discontinuity would be observed in the development of the Hill coefficient with regard to R1. However, the existence of significant heterogeneity among the κ_{[illegible]} values corresponding to the individual microscopic plateaus could lead to a macroscopic discontinuity in the system. This heterogeneity could entail changes in the variation of the Hill coefficient and, as a consequence, the need for a quantitative jump in the macroscopic values of κ so as to ensure that equation (2) fits the data.

Consequently, each plateau may be determined by a different macroscopic resistance constant κ and/or a different macroscopic resistance constant π. The systems in which η=2 can thus be described using a Hill equation in which:

κ takes two different values: κ1 and κ2, depending on the values of R1 under consideration: one part of the curve is described by κ1 while another part of the curve is described by κ2. The Hill curves describing the overall reaction, which curves are characterized by η=2, are hybrids which are generated from two parallel Hill curves which differ solely in the parameter κ. Transition from one curve to the other can change the slope of the resulting Hill curve or

π takes two different values: π1 and π2, depending on the values of R1 under consideration: one part of the curve is described by π1 while another part of the curve is described by π2. The Hill curves describing the overall reaction, which curves are characterized by η=2, are hybrids which are generated from two parallel Hill curves which differ solely in the parameter π. Transition from one curve to the other can alter the slope of the resulting Hill curve.

According to another advantageous embodiment of said methods, the measurement of the assessment parameter ε, representing the specific efficiency of a biological agent which is capable of inducing the production of P in said living target cells, is effected:

either by measuring the slope of the theoretical H curve obtained in (d) at its point of inflexion,

or by calculating the maximum of the first derivative δP/δR1 and, where appropriate, of the second derivative of the theoretical H curve obtained in (d); this is because the efficiency of the reaction described in equation (2) is given by the increase in the output P which can be obtained by increasing the input R1. Thus, the first derivative of P with respect to R1, or the slope of the curve described in equation (2) furnishes the overall efficiency of the reaction for each input or entry R1.

The maximum global efficiency, or ε, is therefore indeed expressed directly, either by the slope at the point of inflexion of the curve described in equation (2) or by the maximum of its derivative δP/δR1. Both the slope of the curve given by equation (2) and the maximum of δP/δR1 increase when ε increases.

According to another advantageous embodiment of said methods, these methods additionally comprise measuring the following parameters: π/Pₘₐₓ, κ/Pₘₐₓ or ε/Pₘₐₓ. These corrected values are independent of the maximum capacity (Pₘₐₓ); they therefore permit a better comparison of the different parameters π, κ and ε when Pₘₐₓ differs depending on the systems or else when the biological agent affects Pₘₐₓ.

According to yet another embodiment of said methods, the values of the Hill parameters corresponding to each biological agent are compared with those obtained using a reference biological agent.

In order to validate the analysis of the reaction R1+R2 by the Hill equation, said methods can, also in accordance with the invention, additionally comprise a step of treating the experimental data obtained in step (d) (Hill plot) in accordance with the following equation:

log|P/(1−P)| vs. log R1.

Also in accordance with the invention, the selection of the biological agents, for example the vectors, which exhibit the minimum acceptable values for the selected parameters: κ, r, ε, θ, τ, η, π/Pₘₐₓ, κ/Pₘₐₓ or ε/Pₘₐₓ can advantageously be subjected to an iterative analysis in order to obtain the H curve which statistically best fits the experimental R1 and P values.

The biological agents which are selected, in accordance with the methods according to the invention, are then definitively validated for their biological properties.

Advantageously, the parameters κ, r, ε, θ, τ, η, π/Pₘₐₓ, κ/Pₘₐₓ or ε/Pₘₐₓ, which are obtained in accordance with the methods according to the invention, are used for:

validating and optimizing the biological agents which can be used in a particular application (method of screening a library of modified biological agents),

developing and optimizing the tests for characterizing biological agents which may possibly have been modified (method of evaluating performance).

By way of example, and in a non-limiting manner, the methods according to the invention can be used for:

screening a library of viral or nonviral vectors for gene therapy, screening a library of antibodies for diagnosing an infection or for selecting antibodies which are effective against tumor cells, or else screening a library of recombinant proteins for diagnosing or treating a human or animal disease;

comparing different vectors or batches of one and the same vector obtained by one and the same preparation method or by different preparation methods.

Alternatively, it is the target cells which are subjected to serial dilutions, with R1 being constant; steps (a) and (b) are therefore modified as a consequence and step (d) comprises the construction, by iteration, of a theoretical H curve which best fits the experimental values (P and R2) by attributing values to the different parameters of the Hill equation (Pₘₐₓ, κ, π and r).

The present invention also relates to a modified biological agent which is characterized in that it can be obtained by the screening method according to the invention.

As well as the above provisions, the invention additionally comprises other provisions which will be evident from the description which follows and which refers to examples of the implementation of the method which is the subject of the present invention and to the appended drawings, in which:

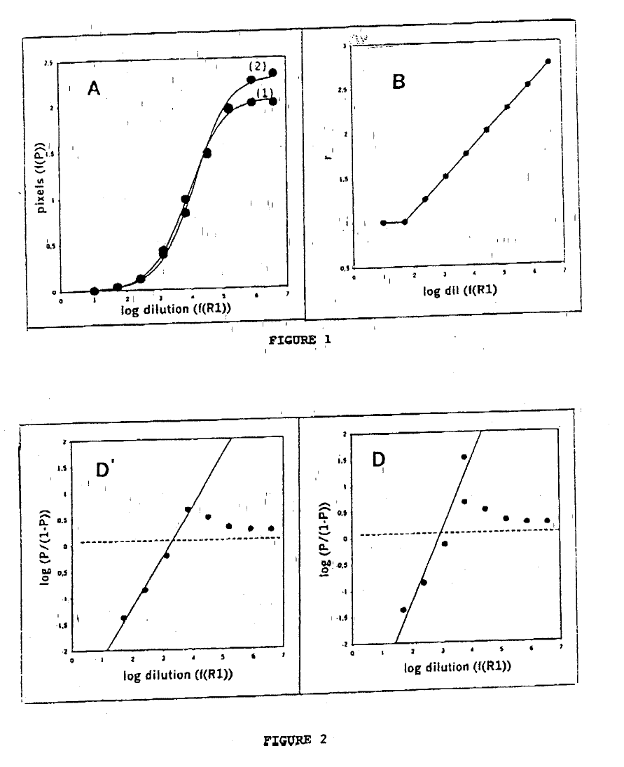

FIG. 1A depicts the theoretical Hill (H) curves which are obtained by iteration from the experimental values P and R1 (f(logdil)) of the two samples 1 and 2 of rAAV. The parameters κ, π, τ and Pₘₐₓ of these two samples were determined directly from these theoretical curves;

FIG. 1B depicts the curve r=f(logdil) which enables the heterogeneity index of the biological system (η) to be determined;

FIG. 2 depicts the Hill plot (log|P/(1−P)|=f(R1));

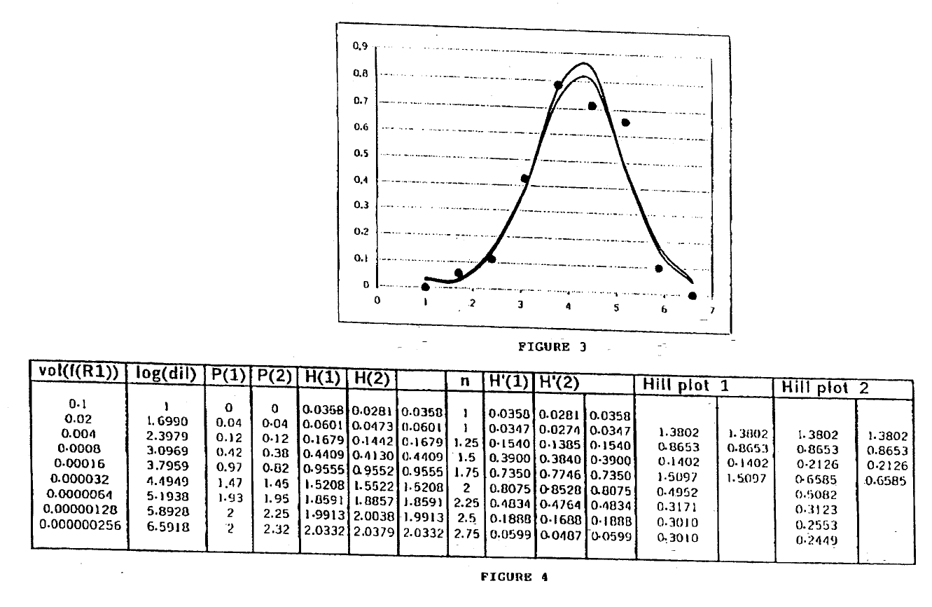

FIG. 3 shows how parameter ε is obtained;

FIG. 4 depicts the experimental values obtained (P and R1 ) and all the calculated theoretical values which were used for drawing the different curves depicted in FIGS. 1 to 3;

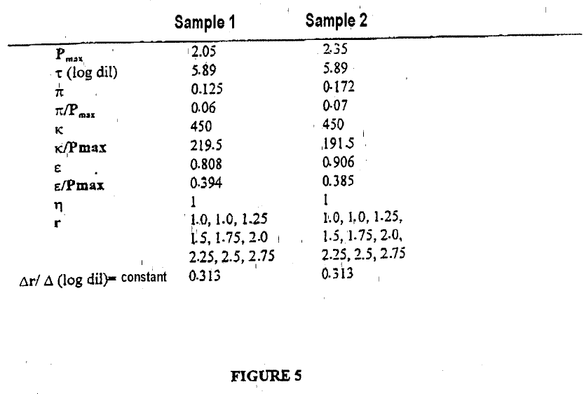

FIG. 5 depicts values of the different Hill parameters which were obtained, in accordance with the invention, for the two samples of rAAV which were studied;

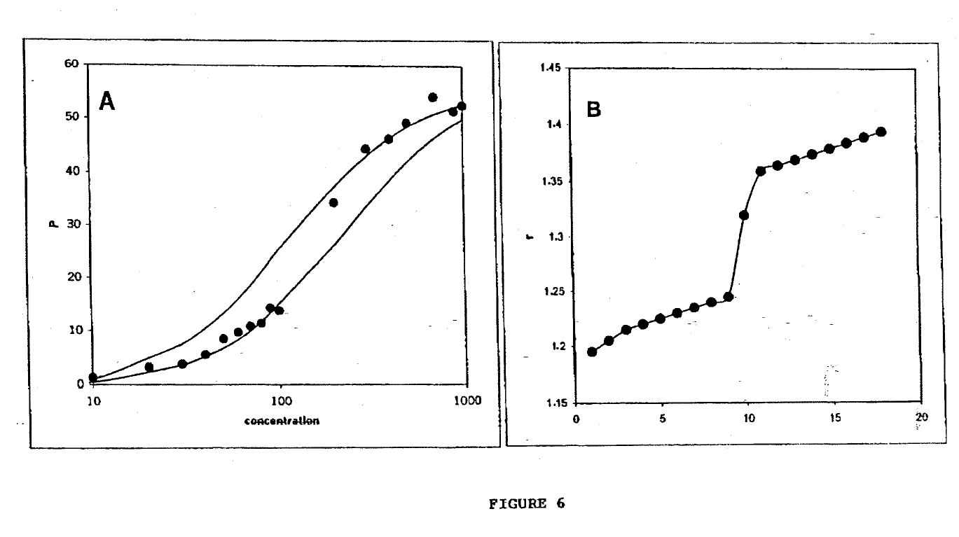

FIGS. 6(A and B) illustrates a heterogeneous biological system (η=2). FIG. 6A depicts the theoretical Hill curves which fit the experimental values of P and R1 (f(concentration)); FIG. 6B comprises, on the x axis, the no. of the point, arranged in accordance with the concentrations in FIG. 6A, and, on the y axis, the values of r; this curve depicts 2 plateaus (η=2);

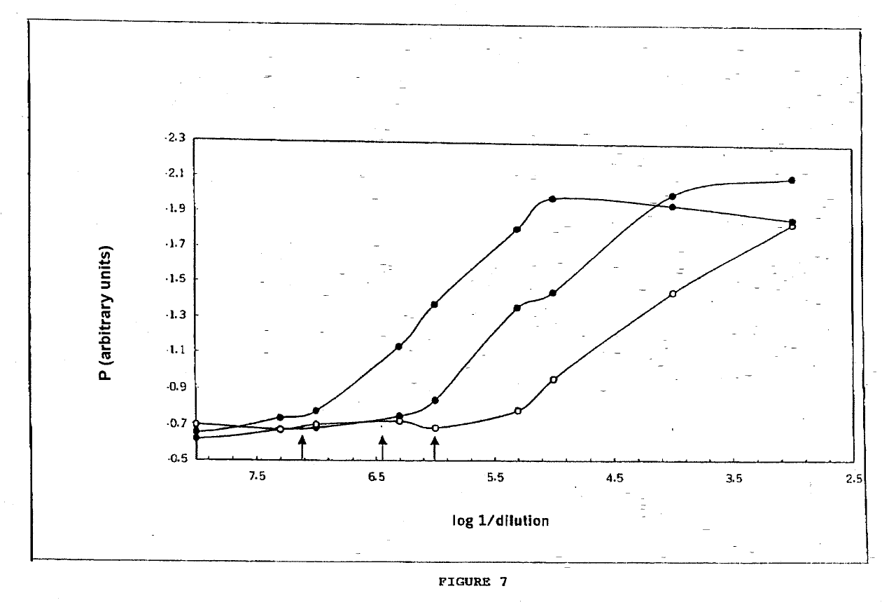

FIG. 7 illustrates the screening of a library of vectors expressing the rAAV rep gene, with each of the vectors encoding a different mutant of said gene. The experimental curves P=f(log 1/dilution) are depicted for each vector and the value of the apparent titer ι, determined from each theoretical Hill curve, is indicated by an arrow on the x axis;

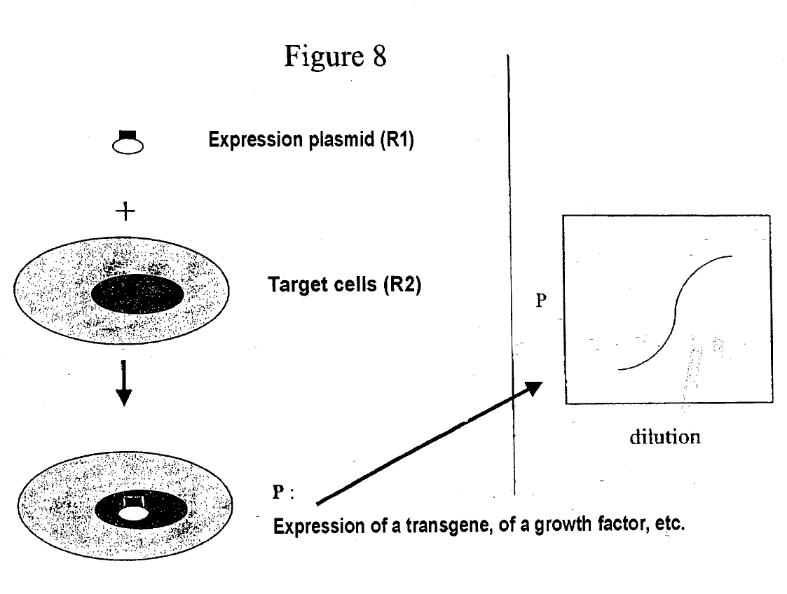

FIG. 8 illustrates the assessment of the performance of an expression plasmid using the method according to the invention; and

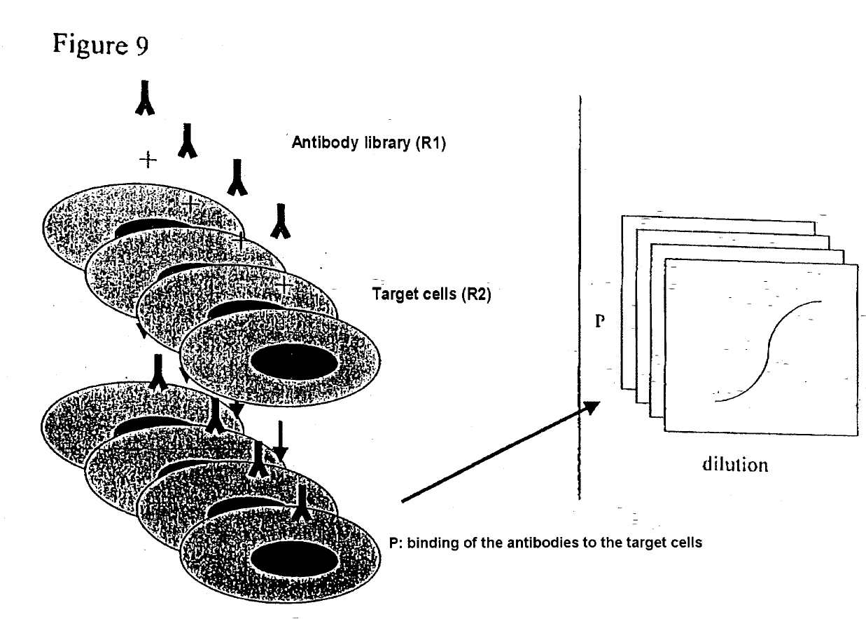

FIG. 9 illustrates the screening of an antibody library by the method according to the invention.

It should of course be understood, however, that these examples are given solely by way of illustrating the subject matter of the invention, to which they in no way constitute a limitation.

### EXAMPLE 1

### Comparison of the Performance of Two Recombinant Vector [Recombinant Adenoassociated Virus (rAAV)] Samples in HeLa rc-32 Cells

a) Materials and Method

Definition of the Biological System

The biological agent studied is a recombinant viral vector (rAAV). Two batches of rAAV (sample 1 and sample 2), which were obtained using standard techniques which are known to the skilled person and described in E. M. Atkinson et al. (NAR, 1998, 26, 11, 2821-2823), were assessed in HeLa rc-32 cells (A. Salvetti et al., Hum. Gene Ther., 1998, 20, 9, 5, 695-706). Cells were sown in the wells of a microtitration plate at a constant concentration R2 and then infected with serial dilutions of samples 1 and 2. At time t=48 h to 72 h, the cells were harvested and the viral genome was then isolated and hybridized with a specific labeled nucleotide probe in accordance with the dot blot technique, which is customarily used by the skilled person and which is described in E. M. Atkinson et al. (NAR, 1998, 26, 11, 2821-2823).

The signal (product P) representing the quantity of DNA hybridized was measured for each dilution of samples 1 and 2 using a phosphorimager, and the values which were obtained are given in FIG. 4. The biological test which was used (hybridization) makes it possible to assess the replication of the biological agent (rAAV) in the HeLa rc-32 target cells.

The steps which were followed, in conformity with the method according to the invention, for analyzing said rAAVs are presented below. All the values of the different Hill parameters which were obtained for the rAAV samples 1 and 2 are compiled in FIG. 5.

Step 1: Determining the Optimum Hill (H) Curve for Each Vector Preparation (FIG. 1A)

The optimum Hill curve, which best fits the experimental values, was obtained by iteration while attributing values to the different parameters of the Hill equation, namely: Pₘₐₓ, κ, π, and r. The best Hill curves which were obtained for samples 1 and 2 are presented in FIG. 1A; they correspond, respectively, to the theoretical values H1 and H2 of samples 1 and 2 (FIG. 4). The values of P are expressed in arbitrary units (pixels) as a function of the log of the dilution of the vector.

The following is obtained in the case of sample 1:

P=2.05 (0.125 R1)^{r}/(450+(0.125 R1)^{r})

The following is obtained in the case of sample 2:

P=2.35 (0.172 R1)^{r}/(450+(0.172 R1)^{r})

*with r=1.0, 1.0, 1.25, 1.5, 1.75, 2.0, 2.25, 2.5, 2.75 *The system used for this test (the HeLa rc-32 cells) is assumed to show the same resistance toward the two samples (κ=450).

Step 2: Plotting the Hill Plot (FIGS. 2A and 2B)

The Hill plot, corresponding to log|P/(1−P)| as a function of the log of the dilution was plotted for the two samples [FIGS. 2D (sample 1) and 2D′ (sample 2)] using the experimental values. The linear regression obtained shows that the data presented in FIG. 1A fit the Hill equation well.

Step 3: Determining the Limiting Concentration (Titer ι) of the Vector Preparations (FIG. 1A)

τ is determined by the maximum value of R1 at which the Hill coefficient (on the optimum Hill curve) is equal to 1. The τ values of samples 1 and 2 were determined from the curves in FIG. 1A; the values obtained are 5.89 for each of the two samples.

Step 4: Determining the Efficiency (ε) and the Standardized Efficiency (ε/Pₘₐₓ) (FIG. 3)

The efficiency is the slope of the Hill curve (or any sigmoidal curve conveying the values obtained) at its point of inflexion.

ε was calculated as follows:

the curve H′ (H′=δH/δR1), derived from H, was plotted.

The maximum value reached by the curve, corresponding to ε, was determined for samples 1 and 2, with the values obtained being 0.808 and 0.906, respectively (FIG. 3).

Step 5: Determining the Homogeneity of the Biological System (η), (FIG. 1B)

The curve r as a function of log dil was plotted and the number of plateaus on the curve was determined.

The heterogeneity index η was determined as follows, as a function of the number of plateaus (one plateau; η=1, two plateaus; η=2, x plateaus; η=x).

η=1 is obtained; consequently, the biological system tested is homogeneous.

Step 6: Complete Characterization of the Vector (FIG. 5)

The set of values obtained for each of the following parameters:

Pₘₐₓ, κ, r, ε, τ, π, η, 68 /Pₘₐₓ and κ/Pₘₐₓ, makes it possible to characterize each vector preparation.

Step 7: Classifying the Vectors in Terms of Their Performance (FIG. 5)

The vectors were classified in terms of the values obtained for:

Pₘₐₓ, κ, π, τ, π/Pₘₐₓ and κ/Pₘₐₓ.

b) Results

The two samples exhibit equivalent apparent titers (ι=5.89); however, the absolute titers are different θ₁/θ₂=π₂/π₁=0.725 and the power of sample 2 (π₁=0.172) is greater than that of sample 1 (π₂=0.125)

This value is not compensated by the corrected power π/Pₘₐₓ since the value found for sample 2 (π/Pₘₐₓ=0.07) is greater than that for sample 1 (π/Pₘₐₓ=0.06).

Considering that κ is constant, that is that the HeLa rc-32 cells exhibit the same resistance toward the two samples, sample 2 is slightly more efficient than sample 1 (ε₂=0.906>ε₁=0.808) and its maximum capacity Pₘₐₓ=2.35 is greater than that of sample 1 (Pₘₐₓ=2.05).

The analysis of the values of the different Hill parameters obtained for the two recombinant vector samples makes it possible to draw the following conclusions:

the apparent titers of two batches of one and the same vector are equivalent and correspond to that which is generally determined in the prior art, whereas determining the Hill parameters and, if necessary, the derived parameters makes it possible to demonstrate differences in their corrected power and their efficiency. Characterizing two batches of one and the same viral vector using relevant Hill parameters and, if necessary, derived Hill parameters, therefore does indeed make it possible to validate and optimize biological agents (preparations of viral vectors, for example) which can be used in a specific application (gene therapy in vivo).

### EXAMPLE 2

### Biological System Which Exhibits a Heterogeneity

a) Defining the Biological System

The biological agent which is analyzed is a retroviral vector, which is designated pSI-EGFP (Ropp et al., Cytometry, 1995, 21, 309-317) and which encodes the eukaryotic fluorescent protein reporter gene (eukaryotic green fluorescent protein or EGFP), and the target cells are HT-1080 cells (ATCC No. CCL-121).

The target cells were seeded at a concentration R2 and infected with serial dilutions of the vector. At time t=48 h, the fluorescence intensity (product P), representing the quantity of the EGFP reporter gene expressed by the vector, was measured for each dilution.

b) Determining the Optimum Hill Curve (FIG. 6A)

The experimental points are not all able to fit a single Hill curve. Two subsets of points are observed:

the points situated in the left-hand part of FIG. 6A fit a Hill curve corresponding to κ=8,000 (upper curve),

the points situated in the right-hand part of FIG. 6A fit a Hill curve corresponding to κ=3,500 (lower curve).

c) Determining the Homogeneity of the Biological System (η), (FIG. 6B)

The curve r as a function of the no. of points, arranged in accordance with the concentrations as they appear in FIG. 6A, was plotted and the number of plateaus on the slope of the curve was determined (FIG. 6B).

The curve exhibits 2 plateaus; consequently, the biological system being tested is heterogeneous and exhibits a heterogeneity index η=2.

### EXAMPLE 3

### Screening a Library of Plasmids Expressing Mutants of the rAAV Rep Gene

a) Materials and Method

Defining the Biological System

The collection of modified biological agents is a library of vectors expressing mutants of the rAAV rep gene and the target cells are an rAAV encapsidation cell line (HeLa rc-32 cell line). The cells were seeded at a constant concentration R2 and then transfected with serial dilutions of the different expression plasmids. At time t=48 h, the quantity of virus produced (product P) was measured by the dot blot technique as described in example 1.

Determining the Hill Parameters

These parameters are determined following steps 1 to 7 as described in example 1, and the final step of selecting the mutant which is most suited to producing the recombinant virus is effected in dependence on the ι values obtained.

b) Results

The experimental curves P=f(log 1/dilution) are depicted in FIG. 7 and the apparent titer ι of each expression plasmid, determined from the theoretical Hill curve, is as follows:

plasmid no. 1 (curve (1)): ι=7.1

plasmid no. 2 (curve (2)): ι=6.4

plasmid no. 3 (curve (3)): ι=6

The results indicate that the mutant of the rAAV rep gene contained in plasmid no. 1 possesses the highest activity in the system tested.

As is evident from the above, the invention is in no way limited to those of its embodiments which have just been described more explicitly; on the contrary, it also encompasses all the variants of which the skilled person can conceive without departing from the context or scope of the present invention.

1. A method for assessing the performance of a collection of complex biological agents in living target cells with which said biological agents interact, which method is characterized in that it comprises at least the following steps:
(a) preparing, for each biological agent in said collection, a range of samples, which range is obtained by making serial dilutions of said biological agent at a concentration R1,
(b) incubating each sample of said range of dilutions obtained in (a) with said target cells at a constant concentration R2,
(c) determining the product P of the reaction R1+R2, at a time t, in each of said samples,
(d) constructing a theoretical H curve from said experimental points R1 and P for each biological agent by iterative approximation of parameters which reflect the reaction R1+R2→P, at said time t, in accordance with the following equation:
P=Pₘₐₓ(πR1)^{r}/(κ+(πR1)^{r}) r=1, . . . , n (2),
in which:
R1 represents the concentration of biological agent in a sample of the range,
P represents the product of the reaction R1+R2 at a time t,
Pₘₐₓ represents the maximum capacity of the reaction,
κ represents the resistance of the living target cells to responding to said biological agent,
r represents a coefficient which depends on R1 and which corresponds to the Hill coefficient, and
π represents the intrinsic power of the biological agent to induce a response in the living target cells, and
(e) sorting the values of κ and π obtained in (d) for each biological agent and classifying the biological agents in accordance with said values.

2. A method for screening a collection of modified complex biological agents (library of mutants) in living target cells with which said biological agents interact, which method is characterized in that it comprises at least the steps (a) to (e) as defined in claim 1 and a step (f) of selecting the biological agent which is most suited for the sought-after application.

3. The method as claimed in claim 1 or claim 2, characterized in that said biological agent is selected from the group comprising viruses, viral and nonviral vectors, vaccines, antibodies and recombinant proteins.

4. The method as claimed in any one of claims 1 to 3, characterized in that P is determined in step (c) either directly, for example by assaying P, or indirectly, for example using a biological test which is appropriately selected for measuring at least one parameter or one variable which reflects the response of the biological system to said biological agent.

5. The method as claimed in any one of claims 1 to 4, characterized in that it additionally comprises measuring at least one of the following derived parameters:
the overall efficiency ε of the reaction induced by the biological agent on said system,
the apparent titer ι of the biological agent, corresponding to the origin of the theoretical H curve obtained in (d),
the absolute titer θ of said biological agent defined by the equation θπ=ι/s (3), in which s represents the sensitivity of the detection method, and
the heterogeneity index η of said biological reaction.

6. The method as claimed in any one of claims 1 to 5, characterized in that the measurement of the assessment parameter ε, representing the specific efficiency of a biological agent which is capable of inducing the production of P in the living target cells, is effected either by measuring the slope of the theoretical curve obtained in (d) at its point of inflexion, or by calculating the maximum of the first derivative and, where appropriate, of the second derivative of the theoretical curve obtained in (d).

7. The method as claimed in any one of claims 1 to 6, characterized in that it additionally comprises measuring the following parameters: π/Pₘₐₓ, κ/Pₘₐₓ or ε/Pₘₐₓ.

8. The method as claimed in any one of claims 1 to 7, characterized in that the values of the Hill parameters corresponding to each biological agent are compared with those obtained using a reference biological agent.

9. The method as claimed in any one of claims 1 to 8, characterized in that, in order to validate the analysis of the reaction R1+R2 by the Hill equation, the method can additionally comprise a step of treating the experimental data obtained in step (d) (Hill plot) in accordance with the following equation:
log|P/(1−P)| vs. log R1.

10. The method as claimed in any one of claims 1 to 9, characterized in that it comprises:
selecting the biological agents which exhibit the minimum acceptable values for the selected parameters: π, κ, r, ε, θ, ι, η, π/Pₘₐₓ, κ/Pₘₐₓ or ε/Pₘₐₓ and
iteratively analyzing the H curves corresponding to said selected biological agents, in accordance with equation (2), in order to eliminate the values raised by compensation, followed by
eliminating the biological agents which compensate and for which alternative curves can be obtained, in order to form a list of biological agents having an optimal action.

11. The method as claimed in any one of claims 2 to 10, characterized in that said collection of modified biological agents comprises a library of mutants which is obtained by naturally or artificially introducing one or more mutations into the nucleotide and/or peptide sequence of said biological agents.

12. A modified biological agent, characterized in that it can be obtained by the method as claimed in claim 11.
